(19) United States
(12) Patent Application Publication
(10) Pub. No.: US 2021/0039983 A1
(43) Pub. Date: Feb. 11, 2021

(54) ARTICLES INCLUDING BRUSHITE FOR USE AS A BONE OR DENTAL IMPLANT AND METHODS OF FORMING

(71) Applicant: CORNING INCORPORATED, CORNING, NY (US)

(72) Inventors: Qiang Fu, Painted Post, NY (US); Michelle Marie Wallen, Hammondsport, NY (US)

(21) Appl. No.: 16/986,524

(22) Filed: Aug. 6, 2020

Related U.S. Application Data
(60) Provisional application No. 62/884,413, filed on Aug. 8, 2019.

(51) Int. Cl. C03C 3/097; A61K 6/58; A61L 27/36; A61L 27/12; A61L 27/02; C03C 23/00; C03C 10/00; A61K 6/838

(52) U.S. Cl. CPC C03C 3/097; A61K 6/58; A61L 27/365; A61L 27/12; C03C 2203/52; C03C 23/00; C03C 10/0009; A61K 6/838; A61L 27/025

(57) ABSTRACT

An article for use as a bone or dental implant and a method of forming said article that includes immersing a silica-based glass substrate in a liquid medium, wherein the liquid medium includes a phosphate source at a concentration of at least about 0.1 moles per liter. The immersing is conducted to convert at least a portion of the silica-based glass substrate into brushite and form the article, wherein the article includes a brushite portion including brushite crystals and a residual glass portion.

### CROSS-REFERENCE TO RELATED APPLICATIONS

This application claims the benefit of priority under 35 U.S.C § 119 of U.S Provisional Application Ser. No. 62/884413 filed on Aug. 8, 2019 the content of which is relied upon and incorporated herein by reference in its entirety.

### FIELD

The present disclosure generally relates to an article including brushite, also referred to as dicalcium phosphate dihydrate (CaHPO₄.2H₂O), for use as a bone or dental implant and methods of forming said article. More specifically, the present disclosure relates to an article formed from a silica-based glass substrate that includes a brushite portion and a residual glass portion.

### BACKGROUND

Calcium-phosphate-based biominerals are used in a variety of medical and dental applications, examples of which include orthopedic implants, dental implants, alveolar ridge augmentation, maxillofacial surgery, otolaryngology, bone regeneration, tooth regeneration, and scaffolds and powders for use in hip and knee surgeries. Hydroxyapatite (Ca₁₀(PO₄)₆(OH)₂) is an example of a calcium-phosphate-based biomineral that is commonly used in medical applications, such as for use as a bone substitute. However, compared to several other calcium-phosphate-based biominerals, hydroxyapatite has a low solubility in most biological environments and thus does not exhibit a high degree of resorbability. For example, dicalcium phosphate dihydrate (CaHPO₄.2H₂O), also referred to as brushite, has a logarithm of thermodynamic solubility in an aqueous solution (log Kₛₚ) of −6.6; α-tricalcium phosphate (α-TCP) has a log Kₛₚ of −25.5; β-tricalcium phosphate (β-TCP) has a log Kₛₚ of −28.9; tetracalcium phosphate (TTCP) has a log Kₛₚ of 38; octacalcium phosphate (OCP) has a log Kₛₚ of 96.6; and hydroxyapatite (HA) has a log Kₛₚ of −116.8. The higher solubility of brushite compared to hydroxyapatite can be desirable in some medical and dental applications, such as in bone and tooth repair, for example.

Conventional methods for forming synthetic brushite include synthesis in either a powder or cement form. Brushite powder can be prepared using conventional methods by a wet chemical synthesis method in which a calcium source, such as CaCl₂·2H₂O, Ca(NO₃)₂·4H₂O, or Ca(CH₃COO)·2H₂O), is combined with a phosphorus source, such as (NH₄)·2HPO₄ or Na₂HPO₄, at a nominal Ca/P molar ratio to precipitate crystals of brushite. The crystals of brushite typically need to undergo additional processing before use, including filtering and ceramic processing steps to form the desired solid structures from the collected crystal powder. Brushite cement can be formed by mixing β-TCP powder with a solution of H₃PO₄ or Ca(H₂PO₄)₂. However, this cement method is typically characterized by a low purity of brushite that can include 10% to 25% of unreacted β-TCP (by weight).

Accordingly, there is a need for an article including brushite, also referred to as dicalcium phosphate dihydrate (CaHPO₄·2H20), for use as a bone or dental implant and methods of forming said article. There is also a need for process that can form high purity brushite crystals that are free, or substantially free, of hydroxyapatite and/or other calcium-phosphate-based biominerals. In addition, there is a need for a process that can form brushite crystals on a scaffold.

### SUMMARY

According to an aspect of the present disclosure, a method of forming an article for use as a bone or dental implant includes immersing a silica-based glass substrate in a liquid medium, wherein the liquid medium includes a phosphate source at a concentration of at least about 0.1 moles per liter. The immersing is conducted to convert at least a portion of the silica-based glass substrate into brushite and form the article, wherein the article includes a brushite portion including brushite crystals and a residual glass portion.

According to an aspect of the present disclosure, an article for use as a dental or bone implant includes a brushite portion present from about 80% to about 99% that includes brushite crystals, and a residual glass portion present from about 1% to about 20% (by weight of the article).

Additional features and advantages will be set forth in the detailed description which follows, and in part will be readily apparent to those skilled in the art from that description or recognized by practicing the aspects as described herein, including the detailed description which follows, the claims, as well as the appended drawings.

It is to be understood that both the foregoing general description and the following detailed description are merely exemplary and are intended to provide an overview or framework to understanding the nature and character of the claims. The accompanying drawings are included to provide a further understanding, and are incorporated in and constitute part of this specification. The drawings illustrate one or more embodiments, and, together with the description, serve to explain the principles and operation of the various embodiments.

### BRIEF DESCRIPTION OF THE DRAWINGS

These and other features, aspects, and advantages of the present disclosure are better understood when the following detailed description of the disclosure is read in reference to the accompanying drawings, in which:

FIG. 1 is a photograph of exemplary articles including a brushite component formed on a glass substrate by immersing the glass substrate in a 0.1 moles per liter K₂HPO₄ solution or a 1.0 moles per liter K₂HPO₄ solution for 60 days according to aspects of the present disclosure and a comparative example glass substrate that was immersed in distilled water for 60 days;

FIG. 2A is a plot of an x-ray powder diffraction analysis of an exemplary article that was formed by immersing Example Glass A in a 1.0 moles per liter K₂HPO₄ solution for 60 days according to an aspect of the present disclosure and a comparative example that includes immersing Example Glass A in distilled water for 60 days;

FIG. 2B is a plot of an x-ray diffraction pattern of an exemplary article that includes immersing Example Glass B in a 1.0 moles per liter K₂HPO₄ solution for 60 days according to an aspect of the present disclosure;

FIG. 2C is a plot of an x-ray diffraction pattern of an exemplary article that was formed by immersing Example Glass A in a 1.0 moles per liter K₂HPO₄ solution 120 days according to an aspect of the present disclosure; and

FIG. 3A is a scanning electron microscope image of an exemplary article that was formed by immersing Example Glass A in a 0.1 moles per liter K₂HPO₄ solution for 120 days at 50× magnification, according to an aspect of the present disclosure;

FIG. 3B is a scanning electron microscope image of the exemplary article of FIG. 3A at 1000× magnification;

FIG. 3C is a scanning electron microscope image of an exemplary article that was formed by immersing Example Glass A in a 1 moles per liter K₂HPO₄ solution for 120 days at 250× magnification, according to an aspect of the present disclosure; and

FIG. 3D is a scanning electron microscope image of the exemplary article of FIG. 3C at 1000× magnification.

### DETAILED DESCRIPTION

In the following detailed description, for purposes of explanation and not limitation, example embodiments disclosing specific details are set forth to provide a thorough understanding of various principles of the present disclosure. However, it will be apparent to one having ordinary skill in the art, having had the benefit of the present disclosure, that the present disclosure may be practiced in other embodiments that depart from the specific details disclosed herein. Moreover, descriptions of well-known devices, methods and materials may be omitted so as not to obscure the description of various principles of the present disclosure. Finally, wherever applicable, like reference numerals refer to like elements.

Unless otherwise expressly stated, it is in no way intended that any method set forth herein be construed as requiring that its steps be performed in a specific order. Accordingly, where a method claim does not actually recite an order to be followed by its steps, or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims, as interpreted according to the principles of patent law, including the doctrine of equivalents.

For purposes of this disclosure, the terms “bulk,” “bulk composition” and/or “overall compositions” are intended to include the overall composition of the entire article, which may be differentiated from a “local composition” or “localized composition” which may differ from the bulk composition owing to the formation of crystalline and/or ceramic phases.

The term “formed from” can mean one or more of comprises, consists essentially of, or consists of. For example, a component that is formed from a particular material can comprise the particular material, consist essentially of the particular material, or consist of the particular material.

As used herein, the terms “article,” “glass-article,” “ceramic-article,” “glass-ceramics,” “glass elements,” “glass-ceramic article” and “glass-ceramic articles” may be used interchangeably, and in their broadest sense, to include any object made wholly or partly of glass and/or glass-ceramic material.

As used herein, a “glass state” refers to an inorganic amorphous phase material within the articles of the disclosure that is a product of melting that has cooled to a rigid condition without crystallizing. As used herein, a “glass-ceramic state” refers to an inorganic material within the articles of the disclosure which includes both the glass state and a “crystalline phase” and/or “crystalline precipitates” as described herein.

As used herein, the language “free” or “substantially free,” when used to describe a constituent of a composition, batch, melt, or article, refers to a constituent that is not actively added, formed, or batched into the composition, batch, melt, or article, but which may be present in a small amount of no more than 1% (by weight) as a contaminant and/or due to the inherent degree of uncertainty attributed to any measurement or analysis technique.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

Aspects of the present disclosure relate to methods for forming dicalcium phosphate dihydrate (CaHPO₄·2H₂O) crystals, also referred to as brushite crystals, from silica-based glass substrates, and articles formed from such methods. The brushite crystals can be formed by immersing a silica-based glass substrate in a liquid medium that includes a phosphate source at a concentration of at least about 0.1 moles per liter, according to aspects of the present disclosure. While not wishing to be limited by any particular theory, the methods of the present disclosure are believed to produce brushite crystals as a result of a chemical reaction that occurs between the silica-based glass substrate and components of the liquid medium when the silica-based glass substrate is immersed in the liquid medium. The chemical reaction with the silica-based glass substrate can result in the growth of a calcium and phosphorus-rich region, which is then converted to brushite.

The brushite component formed when the silica-based glass substrate reacts with components of the liquid medium according to the present disclosure includes a brushite portion that includes brushite crystals and a residual glass portion. In some aspects, the articles of the present disclosure include a brushite portion and a residual glass portion, and optionally a portion of the silica-based glass substrate. In some aspects, the articles of the present disclosure include a brushite portion and a residual glass portion and are free, or substantially free, of unreacted portions of the silica-based glass substrate. Aspects of the methods of the present disclosure can be configured to convert all or a portion of the silica-based glass substrate into a brushite component that includes a brushite portion including brushite crystals and a residual glass portion. The brushite portion may form on or at any surface of the substrate that is exposed to the liquid medium and/or within portions of a body of the substrate that are capable of reacting with components of the liquid medium to form brushite.

The silica-based glass substrate can provide a scaffold upon which the brushite component can form to provide the brushite component in a particular shape based on the intended use of the article. Non-limiting examples of suitable shapes for the silica-based glass substrate include any regular or irregular geometric shape, a spherical shape, or an elongated rod or fiber shape. For example, the silica-based glass substrate can be in the form of glass spheres and the method of the present disclosure can be configured to convert at least a portion of each glass sphere into spheres that include a brushite portion and a residual glass portion, optionally with little to none of the pre-treatment silica-based glass substrate remaining.

The silica-based glass substrate of the present disclosure can include a bioactive glass that is derived from a precursor glass composition that includes SiO₂ and CaO. As used herein, a “bioactive glass” refers to a glass that is biocompatible with bone, tissue, and/or teeth. According to one aspect, the silica-based glass substrate is derived from a precursor glass composition that includes SiO₂, CaO, P₂O₅, and at least one of Na₂O and K₂O. Optionally, the precursor glass composition can include MgO and/or SrO.

The precursor glass composition for the silica-based glass substrate of the present disclosure can include SiO₂ in an amount of at least about 40 wt % (by weight of oxide) and in some aspects, in an amount of from about 40 wt % to about 70 wt % (by weight of oxide). For example, the SiO₂ can be present in an amount of from about 40 wt % to about 70 wt %, about 40 wt % to about 65 wt %, about 40 wt % to about 60 wt %, about 40 wt % to about 55 wt %, about 40 wt % to about 50 wt %, about 40 wt % to about 45 wt %, about 43 wt % to about 47 wt %, about 45 wt % to about 70 wt %, about 45 wt % to about 65 wt %, about 45 wt % to about 60 wt %, about 45 wt % to about 55 wt %, about 45 wt % to about 50 wt %, about 50 wt % to about 70 wt %, about 50 wt % to about 65 wt %, about 50 wt % to about 60 wt %, about 50 wt % to about 55 wt %, about 51 wt % to about 55 wt %, about 55 wt % to about 70 wt %, about 55 wt % to about 65 wt %, about 55 wt % to about 60 wt %, about 60 wt % to about 70 wt %, about 60 wt % to about 65 wt %, or about 65 wt % to about 70 wt % (by weight of oxide). In some examples, the SiO₂ can be present in an amount of about 40 wt %, about 41 wt %, about 42 wt %, about 43 wt %, about 44 wt %, about 45 wt %, about 50 wt %, about 51 wt %, about 52 wt %, about 53 wt %, about 54 wt %, about 55 wt %, about 60 wt %, about 61 wt %, about 62 wt %, about 63 wt %, about 64 wt %, about 65 wt %, about 70 wt % (by weight of oxide), or any amount of SiO₂ between these values.

The precursor glass composition for the silica-based glass substrate can include CaO in an amount of at least about 2 wt %, at least about 5 wt %, at least about 10 wt %, at least about 15 wt %, at least about 20 wt %, or at least about 25 wt % (by weight of oxide). In some aspects, the precursor glass composition can include CaO in an amount of from about 2 wt % to about 30 wt % (by weight of oxide). For example, the CaO can be present in an amount of from about 2 wt % to about 30 wt %, about 2 wt % to about 25 wt %, about 2 wt % to about 20 wt %, about 2 wt % to about 15 wt %, about 2 wt % to about 10 wt %, about 2 wt % to about 5 wt %, about 5 wt % to about 30 wt %, about 5 wt % to about 25 wt %, about 5 wt % to about 20 wt %, about 5 wt % to about 15 wt %, about 5 wt % to about 10 wt %, about 10 wt % to about 30 wt %, about 10 wt % to about 25 wt %, about 10 wt % to about 20 wt %, about 10 wt % to about 15 wt %, about 15 wt % to about 30 wt %, about 15 wt % to about 25 wt %, about 15 wt % to about 20 wt %, about 18 wt % to about 22 wt %, about 20 wt % to about 30 wt %, about 20 wt % to about 25 wt %, about 22.5 wt % to about 26.5 wt %, or about 25 wt % to about 30 wt % (by weight of oxide). In some examples, the CaO can be present in an amount of about 2 wt %, about 3 wt %, about 4 wt %, about 5 wt %, about 10 wt %, about 15 wt %, about 20 wt %, about 25 wt %, about 30 wt % (by weight of oxide), or any amount of CaO between these values.

The precursor glass composition for the silica-based glass substrate can include P₂O₅ in an amount of at least about 2 wt %, at least about 3 wt %, at least about 4 wt %, at least about 5 wt %, at least about 6 wt %, at least about 7 wt %, or at least about 10 wt % (by weight of oxide). In some aspects, the precursor glass composition can include P₂O₅ in an amount of from about 2wt % to about 15 wt % (by weight oxide). For example, the precursor glass composition can include P₂O₅ in an amount of from about 2 wt % to about 15 wt %, about 2 wt % to about 12 wt %, about 2 wt % to about 10 wt %, 2 wt % to about 9 wt %, 2 wt % to about 8 wt %, 2 wt % to about 7 wt %, 2 wt % to about 6 wt %, 2 wt % to about 5 wt %, 2 wt % to about 4 wt %, about 3 wt % to about 15 wt %, about 3 wt % to about 12 wt %, about 3 wt % to about 10 wt %, 3 wt % to about 9 wt %, 3 wt % to about 8 wt %, 3 wt % to about 7 wt %, 3 wt % to about 6 wt %, 3 wt % to about 5 wt %, 3 wt % to about 4 wt %, about 4 wt % to about 15 wt %, about 4 wt % to about 12 wt %, about 4 wt % to about 10 wt %, 4 wt % to about 9 wt %, 4 wt % to about 8 wt %, 4 wt % to about 7 wt %, 4 wt % to about 6 wt %, 4 wt % to about 5 wt %, about 5 wt % to about 10 wt %, 5 wt % to about 9 wt %, 5 wt % to about 8 wt %, 5 wt % to about 7 wt %, 5 wt % to about 6 wt %, about 6 wt % to about 10 wt %, 6 wt % to about 9 wt %, 6 wt % to about 8 wt %, 6 wt % to about 7 wt %, about 7 wt % to about 10 wt %, 7 wt % to about 9 wt %, 7 wt % to about 8 wt %, about 8 wt % to about 10 wt %, or 8 wt % to about 9 wt % (by weight of oxide). In some examples, the precursor glass composition can include P₂O₅ in an amount of about 2 wt %, about 3 wt %, about 4 wt %, about 5 wt %, about 6 wt %, about 7 wt %, about 8 wt %, about 9 wt %, about 10 wt %, about 12 wt %, about 15 wt % (by weight of oxide), or any amount of P₂O₅ between these values.

The precursor glass composition for the silica-based glass substrate can include Na₂O in an amount of from about 2 wt % to about 30 wt % (by weight of oxide). For example, the precursor glass composition can include Na₂O in an amount of from about 2 wt % to about 30 wt %, about 2 wt % to about 25 wt %, about 2 wt % to about 20 wt %, about 2 wt % to about 15 wt %, about 2 wt % to about 10 wt %, about 2 wt % to about 8 wt %, about 2 wt % to about 6 wt %, about 4 wt % to about 30 wt %, about 4 wt % to about 25 wt %, about 4 wt % to about 20 wt %, about 4 wt % to about 15 wt %, about 4 wt % to about 10 wt %, about 4 wt % to about 8 wt %, about 4 wt % to about 6 wt %, about 6 wt % to about 30 wt %, about 6 wt % to about 25 wt %, about 6 wt % to about 20 wt %, about 6 wt % to about 15 wt %, about 6 wt % to about 10 wt %, about 6 wt % to about 8 wt %, about 8 wt % to about 30 wt %, about 8 wt % to about 25 wt %, about 8 wt % to about 20 wt %, about 8 wt % to about 15 wt %, about 8 wt % to about 10 wt %, about 10 wt % to about 30 wt %, about 10 wt % to about 25 wt %, about 10 wt % to about 20 wt %, about 10 wt % to about 15 wt %, about 15 wt % to about 30 wt %, about 15 wt % to about 25 wt %, about 15 wt % to about 20 wt %, about 20 wt % to about 30 wt %, about 20 wt % to about 25 wt %, about 22.5 wt % to about 26.5 wt %, or about 25 wt % to about 30 wt % (by weight of oxide). In some examples, the precursor glass composition can include Na₂O in an amount of about 2 wt %, about 3 wt %, about 4 wt %, about 5 wt %, about 6 wt %, about 7 wt %, about 8 wt %, about 10 wt %, about 12 wt %, about 15 wt %, about 17 wt %, about 20 wt %, about 21 wt %, about 22 wt %, about 23 wt %, about 24 wt %, about 25 wt %, about 30 wt % (by weight of oxide), or any amount of Na₂O between these values.

The precursor glass composition for the silica-based glass substrate can include K₂O in an amount of from about 0 wt % to about 20 wt % (by weight of oxide). For example, the precursor glass composition can include K₂O in an amount of from about 0 wt % to about 20 wt %, about 1 wt % to about 20 wt %, about 2 wt % to about 20 wt %, about 3 wt % to about 20 wt %, about 4 wt % to about 20 wt %, about 5 wt % to about 20 wt %, about 6 wt % to about 20 wt %, about 8 wt % to about 20 wt %, about 10 wt % to about 20 wt %, about 12 wt % to about 20 wt %, about 14 wt % to about 20 wt %, about 16 wt % to about 20 wt %, about 18 wt % to about 20 wt %, about 0 wt % to about 18 wt %, about 1 wt % to about 18 wt %, about 2 wt % to about 18 wt %, about 3 wt % to about 18 wt %, about 4 wt % to about 18 wt %, about 5 wt % to about 18 wt %, about 6 wt % to about 18 wt %, about 8 wt % to about 18 wt %, about 10 wt % to about 18 wt %, about 12 wt % to about 18 wt %, about 14 wt % to about 18 wt %, about 16 wt % to about 18 wt %, about 0 wt % to about 16 wt %, about 1 wt % to about 16 wt %, about 2 wt % to about 16 wt %, about 3 wt % to about 16 wt %, about 4 wt % to about 16 wt %, about 5 wt % to about 16 wt %, about 6 wt % to about 16 wt %, about 8 wt % to about 16 wt %, about 10 wt % to about 16 wt %, about 12 wt % to about 16 wt %, about 0 wt % to about 12 wt %, about 1 wt % to about 12 wt %, about 2 wt % to about 12 wt %, about 3 wt % to about 12 wt %, about 4 wt % to about 12 wt %, about 5 wt % to about 12 wt %, about 6 wt % to about 12 wt %, about 8 wt % to about 12 wt %, about 10 wt % to about 12 wt %, about 0 wt % to about 10 wt %, about 1 wt % to about 10 wt %, about 2 wt % to about 10 wt %, about 3 wt % to about 10 wt %, about 4 wt % to about 10 wt %, about 5 wt % to about 10 wt %, about 0 wt % to about 6 wt %, about 1 wt % to about 6 wt %, about 2 wt % to about 6 wt %, about 3 wt % to about 6 wt %, about 4 wt % to about 6 wt %, about 5 wt % to about 6 wt %, about 0 wt % to about 4 wt %, about 1 wt % to about 4 wt %, about 2 wt % to about 4 wt %, about 3 wt % to about 4 wt %, about 0 wt % to about 2 wt %, or about 1 wt % to about 2 wt % (by weight of oxide). In some examples, the precursor glass composition can include K₂O in an amount of about 0 wt %, about 1 wt %, about 2 wt %, about 3 wt %, about 4 wt %, about 5 wt %, about 6 wt %, about 8 wt %, about 10 wt %, about 12 wt %, about 14 wt %, about 16 wt %, about 18 wt %, about 20 wt % (by weight of oxide), or any amount of K₂O between these values.

In some aspects, a total amount of Na₂O plus K₂O in the precursor glass composition can be at least about 10 wt % (by weight of oxide). For example, the total amount of Na₂O plus K₂O in the precursor glass composition can be at least about 10 wt %, at least about 12 wt %, at least about 18 wt %, at least about 20 wt %, at least about 22 wt %, at least about 25 wt %, or at least about 26 wt % (by weight of oxide). For example, the total amount of Na₂O plus K₂O in the precursor glass composition can be in an amount of from about 10 wt % to about 30 wt %, about 12 wt % to about 30 wt %, about 14 wt % to about 30 wt %, about 16 wt % to about 30 wt %, about 18 wt % to about 30 wt %, about 20 wt % to about 30 wt %, about 22 wt % to about 30 wt %, about 25 wt % to about 30 wt %, about 26 wt % to about 30 wt %, about 10 wt % to about 26 wt %, about 12 wt % to about 26 wt %, about 14 wt % to about 26 wt %, about 16 wt % to about 26 wt %, about 18 wt % to about 26 wt %, about 20 wt % to about 26 wt %, about 22 wt % to about 26 wt %, about 10 wt % to about 22 wt %, about 12 wt % to about 22 wt %, about 14 wt % to about 22 wt %, about 16 wt % to about 22 wt %, about 18 wt % to about 22 wt %, about 20 wt % to about 22 wt %, about 10 wt % to about 20 wt %, about 12 wt % to about 20 wt %, about 14 wt % to about 20 wt %, about 16 wt % to about 20 wt %, about 18 wt % to about 20 wt %, about 10 wt % to about 18 wt %, about 12 wt % to about 18 wt %, or about 14 wt % to about 18 wt % (by weight oxide). In some aspects, the total amount of Na₂O plus K₂O in the precursor glass composition can be about 10 wt %, about 12 wt %, about 14 wt %, about 16 wt %, about 18 wt %, about 20 wt %, about 22 wt %, about 24 wt %, about 26 wt %, about 28 wt %, about 30 wt % (by weight of oxide), or any amount of Na₂O plus K₂O between these ranges.

The precursor glass composition for the silica-based glass substrate can include MgO in an amount of from about 0 wt % to about 10 wt % (by weight of oxide). For example, the precursor glass composition can include MgO in an amount of from about 0 wt % to about 10 wt %, about 2 wt % to about 10 wt %, about 4 wt % to about 10 wt %, about 6 wt % to about 10 wt %, about 8 wt % to about 10 wt %, about 0 wt % to about 8 wt %, about 2 wt % to about 8 wt %, about 4 wt % to about 8 wt %, about 6 wt % to about 8 wt %, about 0 wt % to about 6 wt %, about 2 wt % to about 6 wt %, about 4 wt % to about 6 wt %, about 0 wt % to about 4 wt %, about 2 wt % to about 4 wt %, or about 0 wt % to about 2 wt % (by weight of oxide). In some aspects, the precursor glass composition can include MgO in an amount of about 0 wt %, about 2 wt %, about 3 wt %, about 4 wt %, about 5 wt %, about 6 wt %, about 8 wt %, about 10 wt % (by weight of oxide), or any amount of MgO between these values.

The precursor glass composition for the silica-based glass substrate can include SrO in an amount of from about 0 wt % to about 10 wt % (by weight of oxide). For example, the precursor glass composition can include SrO in an amount of from about 0 wt % to about 10 wt %, about 2 wt % to about 10 wt %, about 4 wt % to about 10 wt %, about 6 wt % to about 10 wt %, about 8 wt % to about 10 wt %, about 0 wt % to about 8 wt %, about 2 wt % to about 8 wt %, about 4 wt % to about 8 wt %, about 6 wt % to about 8 wt %, about 0 wt % to about 6 wt %, about 2 wt % to about 6 wt %, about 4 wt % to about 6 wt %, about 0 wt % to about 4 wt %, about 2 wt % to about 4 wt %, or about 0 wt % to about 2 wt % (by weight of oxide). In some aspects, the precursor glass composition can include SrO in an amount of about 0 wt %, about 2 wt %, about 4 wt %, about 6 wt %, about 8 wt %, about 10 wt % (by weight of oxide), or any amount of SrO between these values.

Exemplary precursor glass compositions for making the silica-based glass substrate according to the present disclosure are shown below in Tables 1-4, identified as “Exemplary Precursor Glass Compositions 1-4.” Tables 1-4 identify the combination of materials and their respective amounts, in ranges, according to the present disclosure. The precursor glass compositions of Tables 1-4 may include additional components according to aspects of the present disclosure discussed herein.

TABLE 1
Exemplary Precursor Glass Composition 1

| | Amount ranges (by weight of oxide) |
|---|---|
| SiO₂ | about 40 wt % to about 70 wt % |
| CaO | about 2 wt % to about 30 wt % |
| P₂O₅ | about 2 wt % to about 15 wt % |
| Na₂O | about 2 wt % to about 30 wt % |
| K₂O | about 0 wt % to about 20 wt % |
| MgO | about 0 wt % to about 10 wt % |
| SrO | about 0 wt % to about 10 wt % |
| Na₂O + K₂O | at least about 10 wt % |

TABLE 2
Exemplary Precursor Glass Composition 2

| | Amount ranges (by weight of oxide) |
|---|---|
| SiO₂ | about 40 wt % to about 70 wt % |
| CaO | about 2 wt % to about 25 wt % |
| P₂O₅ | about 3 wt % to about 10 wt % |
| Na₂O + K₂O | at least about 10 wt % |

TABLE 3
Exemplary Precursor Glass Composition 3

| | Amount ranges (by weight of oxide) |
|---|---|
| SiO₂ | about 43 wt % to about 47 wt % |
| CaO | about 22.5 wt % to about 26.5 wt % |
| P₂O₅ | about 4 wt % to 8 wt % |
| Na₂O | about 22.5 wt % to about 26.5 wt % |

TABLE 4
Exemplary Precursor Glass Composition 4

| | Amount ranges (by weight of oxide) |
|---|---|
| SiO₂ | about 51 wt % to about 55 wt % |
| CaO | about 18 wt % to about 22 wt % |
| P₂O₅ | about 2 wt % to about 6 wt % |
| Na₂O | about 4 wt % to about 8 wt % |
| K₂O | about 10 wt % to about 14 wt % |

The silica-based glass substrates of the present disclosure may be derived from the precursor glass compositions of Tables 1-4, as described above, and formed by any suitable method for forming a glass substrate having the desired dimensions.

Table 5 lists exemplary precursor glass compositions, Exemplary Glass Substrate Precursor Compositions A through I, which can be derived from the precursor glass compositions of Tables 1-4. Table 5 identifies each Exemplary Glass Substrate Precursor Compositions A through I based on the components of the precursor glass composition used to form the substrate (by weight of oxide). The silica-based glass substrates derived from the Exemplary Glass Substrate Precursor Compositions A through I can be used to form the articles of the present disclosure including a brushite portion and a residual glass portion.

TABLE 5
Exemplary Glass Substrate Precursor Compositions A through I

Component

| (% by weight | Exemplary Glass Substrate Precursor Compositions | | | | | | | | |
|---|---|---|---|---|---|---|---|---|---|
| of oxide) | A | B | C | D | E | F | G | H | I |
| SiO₂ | 45 | 53 | 65.5 | 64.3 | 64.3 | 63.2 | 62.1 | 64.3 | 63.2 |
| P₂O₅ | 6 | 4 | 7.4 | 9.1 | 7.3 | 7.1 | 7 | 7.3 | 7.1 |
| Na₂O | 24.5 | 6 | 22.5 | 22.1 | 22.1 | 21.7 | 21.3 | 22.1 | 21.7 |
| K₂O | 0 | 12 | 0 | 0 | 1.8 | 3.6 | 5.3 | 0 | 0 |
| MgO | 0 | 5 | 0 | 0 | 0 | 0 | 0 | 1.8 | 3.6 |
| CaO | 24.5 | 20 | 4.6 | 4.5 | 4.5 | 4.4 | 4.3 | 4.5 | 4.4 |

The brushite portion of the articles of the present disclosure can be present at from about 80 wt % to about 99 wt % of the article. For example, the brushite portion can be present at from about 80 wt % to about 99 wt %, about 85 wt % to about 99 wt %, about 90 wt % to about 99 wt %, about 92 wt % to about 99 wt %, about 95 wt % to about 99 wt %, about 97 wt % to about 99 wt %, about 80 wt % to about 97 wt %, about 85 wt % to about 97 wt %, about 90 wt % to about 97 wt %, about 92 wt % to about 97 wt %, about 95 wt % to about 97 wt %, about 80 wt % to about 95 wt %, about 85 wt % to about 95 wt %, about 90 wt % to about 95 wt %, about 92 wt % to about 95 wt %, about 80 wt % to about 92 wt %, about 85 wt % to about 92 wt %, or about 90 wt % to about 92 wt % of the article. In some examples, the brushite portion can be present at about 80 wt %, about 82 wt %, about 84 wt %, about 86 wt %, about 88 wt %, about 90 wt %, about 92 wt %, about 95 wt %, about 97 wt %, about 99 wt %, or any amount of brushite between these values.

The residual glass portion can be present in the articles of the present disclosure in an amount of from about 1 wt % to about 20 wt % of the article. For example, the residual glass portion can be present at from about 1 wt % to about 20 wt %, about 1 wt % to about 15 wt %, about 1 wt % to about 10 wt %, about 1 wt % to about 5 wt %, about 1 wt % to about 2 wt %, about 2 wt % to about 20 wt %, about 2 wt % to about 15 wt %, about 2 wt % to about 10 wt %, about 2 wt % to about 5 wt %, about 3 wt % to about 20 wt %, about 3 wt % to about 15 wt %, about 3 wt % to about 10 wt %, about 3 wt % to about 5 wt %, about 4 wt % to about 20 wt %, about 4 wt % to about 15 wt %, about 4 wt % to about 10 wt %, about 4 wt % to about 5 wt %, about 5 wt % to about 20 wt %, about 5 wt % to about 15 wt %, about 5 wt % to about 10 wt %, about 10 wt % to about 20 wt %, about 10 wt % to about 15 wt %, or about 15 wt % to about 20 wt %. In some examples, the residual glass portion can be present at about 1 wt %, about 2 wt %, about 3 wt %, about 4 wt %, about 5 wt %, about 6 wt %, about 7 wt %, about 8 wt %, about 9 wt %, about 10 wt %, about 15 wt %, about 20 wt %, or any amount of residual glass between these values. While not wishing to be limited by any theory, it is believed that crystallization at the surface of the silica-based glass substrate can result in a residual glass phase in the brushite component formed from the silica-based glass substrate.

According to one aspect, a calcium-phosphate mineral content of the article is characterized by brushite in an amount of at least about 90 wt % of the article such that the calcium-phosphate mineral content of the article is predominately brushite and is free, or substantially free, of other calcium-phosphate minerals. For example, the calcium-phosphate mineral content of the article can be characterized by brushite present in an amount of at least about 90 wt %, about 91 wt %, about 92 wt %, about 93 wt %, about 94 wt %, about 95 wt %, about 96 wt %, about 97 wt %, about 98 wt %, about 99 wt %, or greater of the article. In one example, the article is free, or substantially free, of a hydroxyapatite portion including hydroxyapatite crystals. As used herein, the article is considered free, or substantially free, of a hydroxyapatite portion including hydroxyapatite crystals, or other calcium-phosphate mineral portion including crystals of the corresponding mineral portion, when such a portion is present at no more than 1 wt % of the article. In this manner, the methods of the present disclosure can produce high purity brushite crystals which are free, or substantially free, of hydroxyapatite crystals. In another example, the article is free, or substantially free, of a hydroxyapatite portion including hydroxyapatite crystals, a α-tricalcium phosphate (α-TCP) portion including α-TCP crystals, a β-tricalcium phosphate (β-TCP) portion including β-TCP crystals, a tetracalcium phosphate (TTCP) portion including TTCP crystals, and/or an octacalcium phosphate (OCP) portion including OCP crystals that is present at no more than 1 wt % of the article.

The brushite crystals in the brushite portion can be characterized by a crystal size of from about 1 micrometer (μm) to about 100 μm. For example, the brushite crystals can be characterized by a crystal size of from about 1 μm to about 100 μm, about 5 μm to about 100 μm, about 10 μm to about 100 μm, about 15 μm to about 100 μm, about 20 μm to about 100 μm, about 25 μm to about 100 μm, about 30 μm to about 100 μm, about 35 μm to about 100 μm, about 40 μm to about 100 μm, about 45 μm to about 100 μm, about 50 μm to about 100 μm, about 55 μm to about 100 μm, about 60 μm to about 100 μm, about 65 μm to about 100 μm, about 70 μm to about 100 μm, about 75 μm to about 100 μm, about 80 μm to about 100 μm, about 85 μm to about 100 μm, about 90 μm to about 100 μm, about 95 μm to about 100 μm, about 5 μm to about 10 μm, about 5 μm to about 20 μm, about 5 μm to about 50 μm, about 5 μm to about 75 μm, about 20 μm to about 50 μm, about 20 μm to about 75 μm, or about 50 μm to about 75 μm. In some examples, the brushite crystals can be characterized by a crystal size of about 1 μm, about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, or any crystal size between these values. Crystal size as used herein is determined by powder x-ray diffraction (XRD) analysis with a scan from 5 to 80 degrees, unless otherwise specified. However, it is understood that different methods and instrumentation for determining crystallite size can also be employed with any necessary scaling. As discussed in more detail below the brushite crystal size can vary depending on aspects of the method used to form the brushite component, non-limiting examples of which include the dimensions of the silica-based glass substrate, a concentration of the phosphate source, a time period the article is immersed in the liquid medium, a temperature of the liquid medium, and a pH of the liquid medium.

The brushite component can have a thickness of from about 1 μm to about 5000 μm or greater. For example, the brushite component can have a thickness of from about 1 μm to about 5000 μm, about 1 μm to about 2500 μm, about 1 μm to about 1000 μm, about 1 μm to about 750 μm, about 1 μm to about 500 μm, about 1 μm to about 250 μm, about 1 μm to about 100 μm, about 100 μm to about 5000 μm, about 100 μm to about 2500 μm, about 100 μm to about 1000 μm, about 100 μm to about 750 μm, about 100 μm to about 500 μm, about 100 μm to about 250 μm, about 250 μm to about 5000 μm, about 250 μm to about 2500 μm, about 250 μm to about 1000 μm, about 250 μm to about 750 μm, about 250 μm to about 500 μm, about 500 μm to about 5000 μm, about 500 μm to about 2500 μm, about 500 μm to about 1000 μm, about 500 μm to about 750 μm, about 1000 μm to about 5000 μm, or about 2500 μm to about 5000 μm. In one example, the brushite component can have a thickness of about 1 μm, about 50 μm, about 100 μm, about 250 μm, about 500 μm, about 750 μm, about 1000 μm, about 2500 μm, about 5000 μm, or any thickness between these values. As discussed in more detail below the thickness of the brushite component can vary depending on aspects of the method used to form the brushite component, non-limiting examples of which include the dimensions of the silica-based glass substrate, a concentration of the phosphate source, a time period the article is immersed in the liquid medium, a temperature of the liquid medium, and a pH of the liquid medium.

A method according to the present disclosure for forming the articles of the present disclosure including a brushite portion and a residual glass portion are now described. The method includes immersing the silica-based glass substrate in a liquid medium that includes a phosphate source at a concentration of at least about 0.1 moles per liter. The liquid medium can include any suitable source of phosphate that can provide a source of phosphorus for reacting with the silica-based glass substrate to form brushite crystals. Non-limiting examples of suitable sources of phosphate include phosphoric acid (H₃PO₄), monosodium phosphate (NaH₂PO₄), monopotassium phosphate (KH₂PO₄), dipotassium phosphate (K₂HPO₄), ammonium dihydrogen phosphate (NH₄H₂PO₄), or any other phosphate salt. The liquid medium can include one or more solvents, an example of which includes water and distilled water, in which the phosphate source is soluble and can be dissolved.

The concentration of the phosphate source can be at least about 0.1 moles per liter and in some aspects the concentration of the phosphate source is from about 0.1 moles per liter to about 5 moles per liter. The concentration of the phosphate source can be selected to provide the brushite component with the desired characteristics, such as a brushite crystal size, a rate of brushite crystal formation, and a thickness of the brushite component formed from the silica-based glass substrate, for example. For example, the concentration of the phosphate source can be at least about 0.1 moles per liter, at least about 0.2 moles per liter, at least about 0.3 moles per liter, at least about 0.4 moles per liter, at least about 0.5 moles per liter, at least about 0.6 moles per liter, at least about 0.7 moles per liter, at least about 0.8 moles per liter, at least about 0.9 moles per liter, at least about 1 moles per liter, at least about 1.25 moles per liter, at least about 1.5 moles per liter, at least about 1.75 moles per liter, at least about 2 moles per liter, at least about 2.25 moles per liter, at least about 2.5 moles per liter, at least about 2.75 moles per liter, at least about 3 moles per liter, at least about 3.25 moles per liter, at least about 3.5 moles per liter, at least about 3.75 moles per liter, at least about 4 moles per liter, at least about 4.25 moles per liter, at least about 4.5 moles per liter, at least about 4.75 moles per liter, or at least about 5 moles per liter. In some examples, the concentration of the phosphate source can be from about 0.1 moles per liter to about 5 moles per liter, about 0.1 moles per liter to about 4.75 moles per liter, about 0.1 moles per liter to about 4.5 moles per liter, about 0.1 moles per liter to about 4.25 moles per liter, about 0.1 moles per liter to about 4 moles per liter, about 0.1 moles per liter to about 3.75 moles per liter, about 0.1 moles per liter to about 3.5 moles per liter, about 0.1 moles per liter to about 3.25 moles per liter, about 0.1 moles per liter to about 3 moles per liter, about 0.1 moles per liter to about 2.75 moles per liter, about 0.1 moles per liter to about 2.5 moles per liter, about 0.1 moles per liter to about 2.25 moles per liter, about 0.1 moles per liter to about 2 moles per liter, about 0.1 moles per liter to about 1.75 moles per liter, about 0.1 moles per liter to about 1.5 moles per liter, about 0.1 moles per liter to about 1.25 moles per liter, about 0.1 moles per liter to about 1 moles per liter, about 0.1 moles per liter to about 0.75 moles per liter, or about 0.1 moles per liter to about 0.5 moles per liter. In some examples, the concentration of the phosphate source can be about 0.1 moles per liter, about 0.2 moles per liter, about 0.3 moles per liter, about 0.4 moles per liter, about 0.5 moles per liter, about 0.6 moles per liter, about 0.7 moles per liter, about 0.8 moles per liter, about 0.9 moles per liter, about 1 moles per liter, about 1.25 moles per liter, about 1.5 moles per liter, about 1.75 moles per liter, about 2 moles per liter, about 2.25 moles per liter, about 2.5 moles per liter, about 2.75 moles per liter, about 3 moles per liter, about 3.25 moles per liter, about 3.5 moles per liter, about 3.75 moles per liter, about 4 moles per liter, about 4.25 moles per liter, about 4.5 moles per liter, about 4.75 moles per liter, about 5 moles per liter, or any concentration of the phosphate source between these values. In some aspects, an upper limit on the concentration of the phosphate source may be based on the ability to dissolve the phosphate source in the solvent of the liquid medium. In some examples, the solvent may be heated to facilitate dissolving the phosphate source in the solvent, and then the liquid medium may optionally be allowed to cool to a predetermined temperature prior to immersing the silica-based glass substrate in the liquid medium.

The silica-based glass substrate may remain immersed in the liquid medium for a time period of from about 1 day to about 120 days. The immersion time period may be selected based on a desired degree of conversion of the silica-based glass substrate to brushite. For example, as the immersion time period is increased, a degree or extent of the conversion of the silica-based glass substrate to brushite, which can be measured based on a thickness of the brushite component formed from the silica-based glass substrate, will also increase. For example, when all other treatment factors are the same, when a thicker layer of brushite is desired (i.e., an increase in the extent of the conversion of the silica-based substrate to brushite), the immersion time period can be increased and when a thinner layer of brushite is desired (i.e., a decrease in the extent of the conversion of the silica-based substrate to brushite), the immersion time period can be decreased. For example, the time period of immersion of the silica-based glass substrate in the liquid medium can be from about 1 day to about 120 days, about 1 day to about 100 days, about 1 day to about 80 days, about 1 day to about 60 days, about 1 day to about 40 days, about 1 day to about 20 days, about 1 day to about 10 days, about 1 day to about 5 days, about 5 days to about 120 days, about 5 days to about 100 days, about 5 days to about 80 days, about 5 days to about 60 days, about 5 days to about 40 days, about 5 days to about 20 days, about 5 days to about 10 days, about 10 days to about 120 days, about 10 days to about 100 days, about 10 days to about 80 days, about 10 days to about 60 days, about 10 days to about 40 days, about 10 days to about 20 days, about 20 days to about 120 days, about 20 days to about 100 days, about 20 days to about 80 days, about 20 days to about 60 days, about 20 days to about 40 days, about 40 days to about 120 days, about 40 days to about 100 days, about 40 days to about 80 days, about 40 days to about 60 days, about 60 days to about 120 days, about 60 days to about 100 days, about 60 days to about 80 days, about 80 days to about 120 days, or about 80 days to about 100 days. In some examples, the time period of immersion of the silica-based glass substrate in the liquid medium can be about 1 day, about 2 days, about 5 days, about 10 days, about 15 days, about 20 days, about 30 days, about 40 days, about 50 days, about 55 days, about 60 days, about 70 days, about 80 days, about 90 days, about 100 days, about 110 days, about 120 days, or any number of days between these values.

A temperature of the liquid medium can be from about 10° C. to about 85° C. In one example, the method can be performed at room temperature without any active heating or cooling. In other examples, the liquid medium may be actively heated and/or cooled to maintain the liquid medium at a predetermined temperature. For example, the temperature of the liquid medium can be from about 10° C. to about 85° C., about 10° C. to about 80° C., about 10° C. to about 75° C., about 10° C. to about 70° C., about 10° C. to about 65° C., about 10° C. to about 60° C., about 10° C. to about 55° C., about 10° C. to about 50° C., about 10° C. to about 45° C., about 10° C. to about 40° C., about 10° C. to about 35° C., about 10° C. to about 30° C., about 10° C. to about 25° C., about 10° C. to about 20° C., about 20° C. to about 85° C., about 20° C. to about 80° C., about 20° C. to about 75° C., about 20° C. to about 70° C., about 20° C. to about 65° C., about 20° C. to about 60° C., about 20° C. to about 55° C., about 20° C. to about 50° C., about 20° C. to about 45° C., about 20° C. to about 40° C., about 20° C. to about 35° C., about 20° C. to about 30° C., about 20° C. to about 25° C., about 25° C. to about 85° C., about 25° C. to about 80° C., about 25° C. to about 75° C., about 25° C. to about 70° C., about 25° C. to about 65° C., about 25° C. to about 60° C., about 25° C. to about 55° C., about 25° C. to about 50° C., about 25° C. to about 45° C., about 25° C. to about 40° C., about 25° C. to about 35° C., about 25° C. to about 30° C., about 30° C. to about 85° C., about 30° C. to about 80° C., about 30° C. to about 75° C., about 30° C. to about 70° C., about 30° C. to about 65° C., about 30° C. to about 60° C., about 30° C. to about 55° C., about 30° C. to about 50° C., about 30° C. to about 45° C., about 30° C. to about 40° C., about 30° C. to about 35° C., about 40° C. to about 85° C., about 40° C. to about 80° C., about 40° C. to about 75° C., about 40° C. to about 70° C., about 40° C. to about 65° C., about 40° C. to about 60° C., about 40° C. to about 55° C., about 40° C. to about 50° C., about 50° C. to about 85° C., about 50° C. to about 80° C., about 50° C. to about 75° C., about 50° C. to about 70° C., about 50° C. to about 65° C., about 50° C. to about 60° C., about 60° C. to about 85° C., about 60° C. to about 80° C., about 60° C. to about 75° C., about 60° C. to about 70° C., about 70° C. to about 85° C., or about 70° C. to about 80° C. In some examples, the temperature of the liquid medium is about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 30° C., about 35° C., about 40° C., about 45° C., about 50° C., about 55° C., about 60° C., about 65° C., about 70° C., about 75° C., about 80° C., about 85° C., or any temperature between these values.

Optionally, the liquid medium can include one or more additional components to facilitate growth of the brushite component, such as one or more salts and/or buffers. In one example, the liquid medium can include one or more buffers, such as a tris(hydroxymethyl)aminomethane buffer, for example, to adjust the pH of the liquid medium to facilitate growth of the brushite crystals.

At the end of the immersion time period, the article can be removed from the liquid medium, optionally rinsed, and then dried. The rinsing liquid can include any suitable solvent, examples of which include water, alcohol (e.g., ethanol), or a mixture thereof In one example, the article can be allowed to dry in air, at room temperature (e.g., about 20° C. to about 25° C.). Optionally, the brushite component can be removed from any remaining portion of the silica-based glass substrate and stored in powder form for testing and/or later use. In another example, the article can be used as formed without separating the brushite component from any underlying silica-based glass substrate that may remain.

One or more aspects of the method can be selected to provide an article having the desired characteristics within a desired period of time. According to aspects of the present disclosure, a concentration of the phosphate source, a time period the article is immersed in the liquid medium, a temperature of the liquid medium, and a pH of the liquid medium can all be selected in concert with the materials and dimensions of the silica-based glass substrate to provide an article including a brushite portion and a residual glass portion having the desired characteristics based on the intended use of the article. For example, in some aspects, a concentration of the phosphate source can be selected to provide brushite crystals having a crystal size within a predetermined range. In some examples, with all other experimental factors being equal, a lower concentration of the phosphate source, such as 0.1 moles per liter, for example, can produce a higher proportion of course grain brushite crystals (e.g., crystal size of about 50 μm to about 100 μm), whereas higher concentrations, such as 1 mole per liter, for example, can produce a higher proportion of fine grain brushite crystals (e.g., crystal size of about 5 μm to about 20 μm).

While not wishing to be limited by any particular theory, the methods of the present disclosure are believed to produce brushite crystals as a result of a chemical reaction that occurs at a surface of the silica-based glass substrate when the silica-based substrate is immersed in the liquid medium. The chemical reaction at the surface of the silica-based glass substrate can result in the growth of a calcium and phosphorus-rich region, which is then converted to brushite. The source of calcium for forming the brushite component can include calcium present in the silica-based glass substrate. The source of phosphorus for forming the brushite component can include phosphorus present in the silica-based glass substrate and/or phosphorus present in the liquid medium. The initial reactions believed to be part of the reaction process that ultimately forms the brushite may include an exchange of alkali ions in the silica-based glass substrate with hydronium ions present in the liquid medium, which can result in the formation of Si(OH)₄ and then Si—OH (silanol) in the silica-based glass substrate. Further reaction of the silanol produces a SiO₂-rich region that can provide a nucleation site for the deposition of calcium and phosphorus ions, which form the calcium and phosphorus-rich region, and which can be subsequently converted to brushite.

Hydroxyapatite has a lower aqueous solubility than brushite and thus it would be expected that hydroxyapatite would form at the surface of the silica-based glass substrate. However, the methods of the present disclosure are configured to brushite crystals that are free, or substantially free, of hydroxyapatite crystals. In some aspects, the methods of the present disclosure are capable of producing brushite that is free, or substantially free, of a hydroxyapatite portion including hydroxyapatite crystals, a α-tricalcium phosphate (α-TCP) portion including α-TCP crystals, a β-tricalcium phosphate (β-TCP) portion including β-TCP crystals, a tetracalcium phosphate (TTCP) portion including TTCP crystals, and/or an octacalcium phosphate (OCP) portion including OCP crystals that is present at no more than 1 wt % of the article. In this manner, the methods of the present disclosure can be considered as producing high purity brushite crystals that are free, or substantially free, of other calcium-phosphate mineral crystals, such as hydroxyapatite, a-tricalcium phosphate (α-TCP), β-tricalcium phosphate (β-TCP), tetracalcium phosphate (TTCP), and/or octacalcium phosphate (OCP).

### EXAMPLE

The following example describe various features and advantages provided by the embodiments of the disclosure, and is in no way intended to limit aspects of the present disclosure and appended claims.

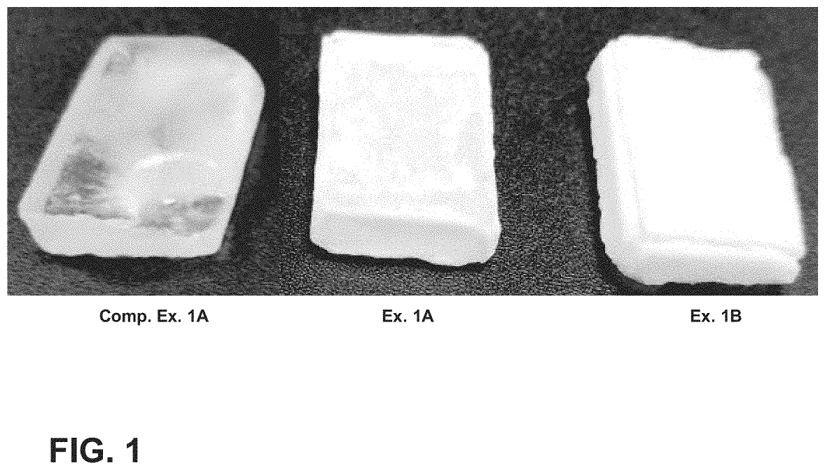

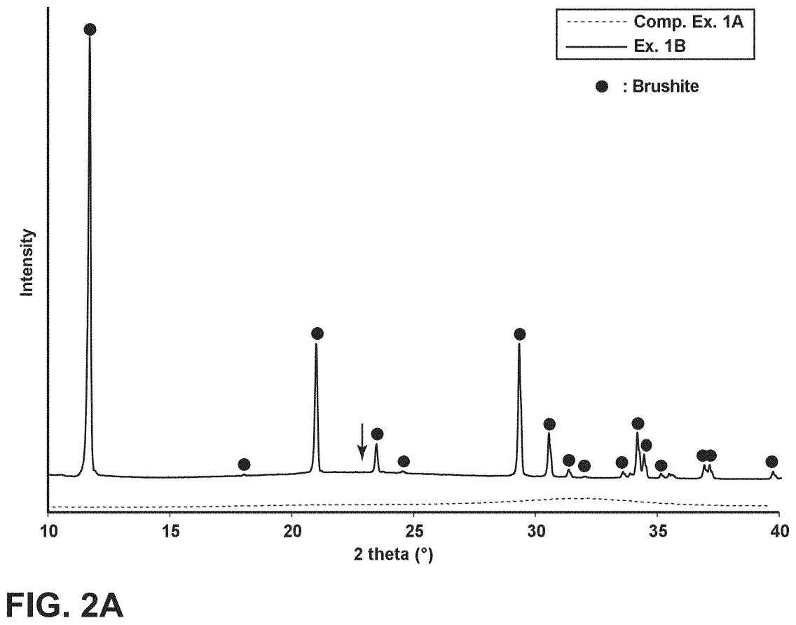

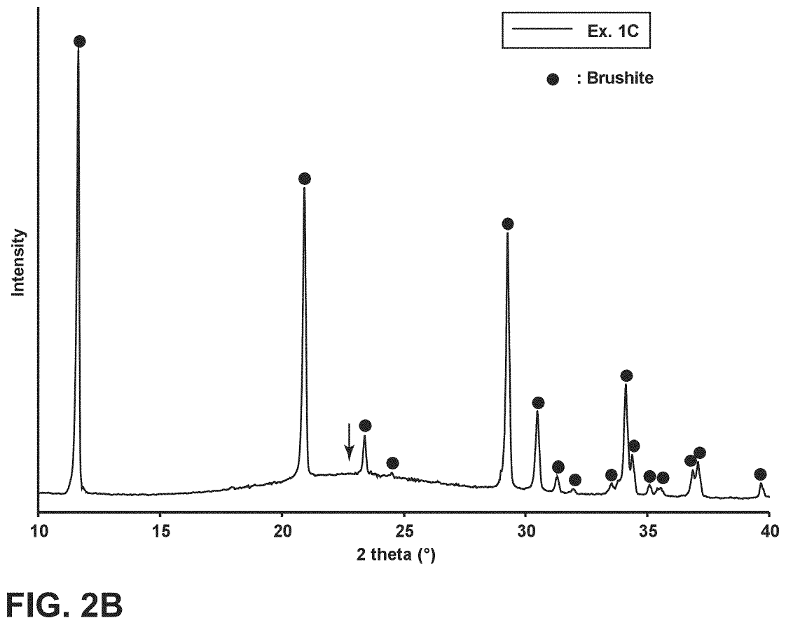

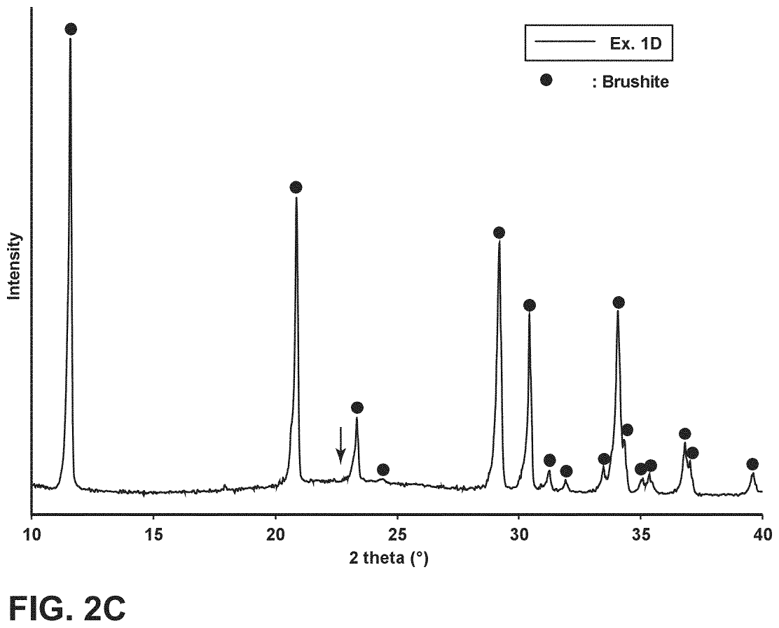

Referring now to FIGS. 1, 2A-C, and 3A-D, exemplary and comparative samples were prepared by immersing silica-based glass substrates in a liquid medium as indicated in Table 6 below. The silica-based glass substrates used were derived from either Exemplary Glass Substrate Precursor Composition A or B from Table 5 above. The liquid medium for Examples 1A-1E (“Ex. 1A-1E”) included K₂HPO₄ as the phosphate source dissolved in distilled water at appropriate amounts to provide the concentrations indicated in Table 6 below. The liquid medium for the Comparative Example 1A (“Comp. Ex. 1A”) was distilled water. The dimensions of the silica-based glass substrates were approximately 25.4 mm by 19 mm by 6 mm. The samples were immersed in the liquid medium for the time period indicated below in Table 6 and then removed, rinsed with ethanol, and allowed to dry in air, at room temperature. The samples were then analyzed as described below.

TABLE 6
Exemplary Examples 1A-1E and Comparative Example 1A

| | Exemplary Glass | | | |
|---|---|---|---|---|
| | Substrate Precursor | Phosphate source | | Time period |
| | Composition | concentration | | (days) |
| Ex. 1A | A | 0.1 | moles/liter | 60 |
| Ex. 1B | A | 1 | mole/liter | 60 |
| Ex. 1C | B | 1 | mole/liter | 60 |
| Ex. 1D | A | 0.1 | moles/liter | 120 |
| Ex. 1E | A | 1 | mole/liter | 120 |

| | | | |
|---|---|---|---|
| Comp. Ex. 1A | A | 0 | 60 |

As illustrated by the photograph shown in FIG. 1, Ex. 1A and Ex. 1B demonstrate that a material is formed on the silica-based glass substrate that is white in appearance when the silica-based glass substrate is immersed in a liquid medium including a phosphate source according to the present disclosure. In contrast, Comp. Ex. 1A, which was immersed only in distilled water, does not show any visible evidence of the formation of a white material on the silica-based glass substrate.

FIGS. 2A-C illustrate the x-ray diffraction (XRD) pattern of the material formed on the silica-based glass substrate when treated according to the aspects of the present disclosure. Samples were prepared for Ex. 1B, Ex. 1C, and Ex. 1D, as described above. A portion of the material formed following immersion of the silica-based glass substrate in the liquid medium, as indicated in Table 6 above, was removed from the silica-based glass substrate and collected for XRD analysis (after the samples had been rinsed with ethanol and dried in air).

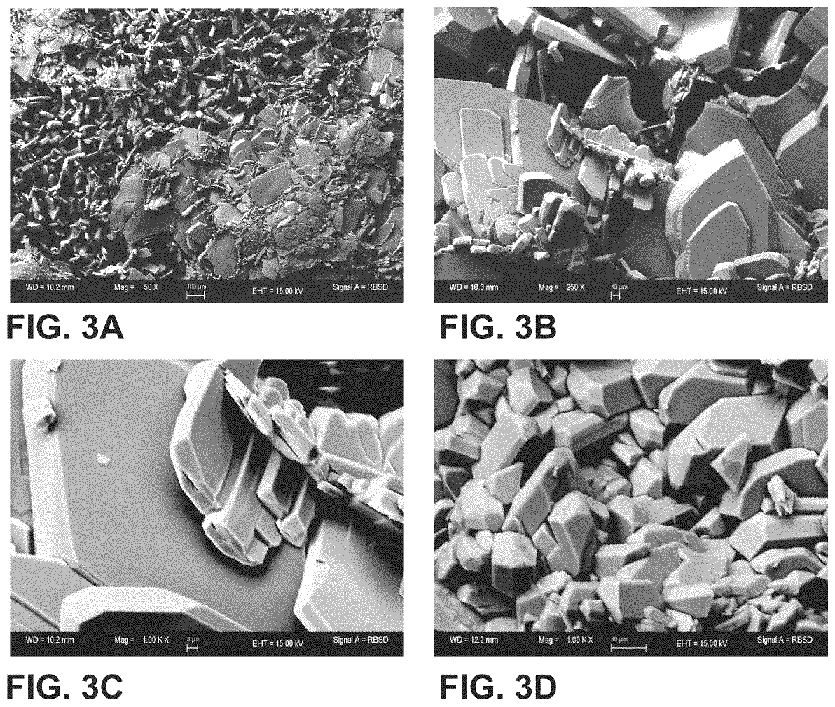

As illustrated in FIGS. 2A-C, only peaks indicative of brushite are apparent in the XRD pattern for Ex. 1B, Ex. 1C, and Ex. 1D, respectively. The XRD data does not show any peaks indicative of hydroxyapatite or other calcium-phosphate minerals, α-tricalcium phosphate (α-TCP), β-tricalcium phosphate (β-TCP), tetracalcium phosphate (TTCP), and/or octacalcium phosphate (OCP). The XRD data for Ex. 1B, Ex. 1C, and Ex. 1D illustrate the ability of the methods of the present disclosure to form brushite that is free, or substantially free, of other calcium-phosphate minerals, such as hydroxyapatite, α-tricalcium phosphate (α-TCP), β-tricalcium phosphate (β-TCP), tetracalcium phosphate (TTCP), and/or octacalcium phosphate (OCP). The halo visible in the XRD data of FIGS. 2A-C, generally indicated by an arrow in the figure, is indicative of a residual glass portion that is part of the material formed according to the present disclosure. The XRD data of FIGS. 2A-C illustrate the ability of the methods of the present disclosure to form a brushite component that includes a brushite portion including brushite crystals and a residual glass portion, and which is free, or substantially free, of other calcium-phosphate minerals, such as hydroxyapatite, α-tricalcium phosphate (α-TCP), β-tricalcium phosphate (β-TCP), tetracalcium phosphate (TTCP), and/or octacalcium phosphate (OCP). The data of FIGS. 3A-C also illustrate the ability to form high purity brushite on different glass substrates, with different concentrations of phosphate sources, and different immersion time periods.

FIGS. 3A-D illustrate scanning electron microscope (SEM) data for samples made according to Ex. 2D and Ex. 2E. FIGS. 3A and 3B show a 50× and 1000× magnification, respectively, of an SEM image of brushite crystals of Ex. 2D. The average crystal size for Ex. 2D was about 50 μm to about 100 μm. FIGS. 3C and 3D show a 250× and 1000× magnification, respectively, of an SEM image of brushite crystals of Ex. 2E. The average crystal size for Ex. 2E was about 5 μm to about 20 μm. Ex. 2E was made using a phosphate concentration of 1 mole/liter, which is 10 times the phosphate concentration of Ex. 2D (0.1 moles/liter). The data demonstrate that treatment with a higher concentration of the phosphate source can produce a finer grain brushite crystal than a lower concentration of the phosphate source. This data indicates that features of the method of the present disclosure, such as the concentration of the phosphate source, can be used to control the size of the brushite crystals formed.

The following non-limiting aspects are encompassed by the present disclosure:

According to a first aspect of the present disclosure, a method of forming an article for use as a bone or dental implant, includes: immersing a silica-based glass substrate in a liquid medium, wherein the liquid medium includes a phosphate source at a concentration of at least about 0.1 moles per liter, further wherein the immersing is conducted to convert at least a portion of the silica-based glass substrate into brushite and form the article, wherein the article includes a brushite portion including brushite crystals and a residual glass portion.

According to a second aspect, the method according to the first aspect, wherein the article includes from about 1% to about 20% of the residual glass portion (by weight of the article).

According to a third aspect, the method according to the first or second aspects, wherein the phosphate source includes at least one of phosphoric acid, monosodium phosphate, dipotassium phosphate, monopotassium phosphate, and ammonium dihydrogen phosphate.

According to a fourth aspect, the method of any one of the first to the third aspects, wherein the liquid medium includes a phosphate source at a concentration of from about 0.1 moles per liter to about 5 moles per liter.

According to a fifth aspect, the method of any one of the first to the fourth aspects, wherein the step of immersing a silica-based glass substrate is conducted for a period of from about 1 day to about 120 days.

According to a sixth aspect, the method of any one of the first to the fifth aspects, wherein the brushite portion includes a plurality of brushite crystals having a crystal size of from about 1 micrometer to about 100 micrometers.

According to a seventh aspect, the method of any one of the first to the sixth aspects, wherein the liquid medium is at a temperature of from about 10° C. to about 85° C. during the step of immersing a silica-based glass substrate.

According to an eighth aspect, the method of any one of the first to the seventh aspects, wherein the silica-based glass substrate is derived from a precursor glass composition including at least about 2% calcium oxide (by weight of oxide).

According to a ninth aspect, the method of any one of the first to the eighth aspects, wherein the silica-based glass substrate is derived from a precursor glass composition including at least about 3% phosphorous pentoxide (by weight of oxide).

According to a tenth aspect, the method of any one of the first to the ninth aspects, wherein the silica-based glass substrate is derived from a precursor glass composition including at least about 40% silicon dioxide (by weight of oxide).

According to an eleventh aspect, the method of any one of the first to the seventh aspects, wherein the silica-based glass substrate is derived from a precursor glass composition including: SiO₂ from about 40% to about 70%, CaO from about 2% to about 25%, P₂O₅ from about 3% to about 10%, and a total amount of Na₂O plus K₂O of at least about 10% (by weight of oxide).

According to a twelfth aspect, the method of any one of the first to the seventh aspects, wherein the silica-based glass substrate is derived from a precursor glass composition including: SiO₂ from about 43% to about 47%, CaO from about 22.5% to about 26.5%, P₂O₅ from about 4% to about 8%, and Na₂O from about 22.5% to about 26.5% (by weight of oxide).

According to a thirteenth aspect, the method of any one of the first to the seventh aspects, wherein the silica-based glass substrate is derived from a precursor glass composition including: SiO₂ from about 51% to about 55%, CaO from about 18% to about 22%, P₂O₅ from about 2% to about 6%, K₂O from about 10% to about 14%, and Na₂O from about 4% to about 8% (by weight of oxide).

According to a fourteenth aspect, the method of any one of the first to the seventh aspects, wherein the silica-based glass substrate is derived from a precursor glass composition including: SiO₂ from about 40% to about 70%, CaO from about 2% to about 30%, P₂O₅ from about 2% to about 15%, MgO from about 0% to about 10%, SrO from about 0% to about 10%, and a total amount of Na₂O plus K₂O of at least about 10% (by weight of oxide).

According to the fifteenth aspect, the method of any one of the first to the fourteenth aspects, wherein the article is free, or substantially free, of a hydroxyapatite portion including hydroxyapatite crystals at no more than 1% of the article (by weight).

According to a sixteenth aspect of the present disclosure, an article for use as a dental or bone implant, includes: a brushite portion that includes brushite crystals present from about 80% to about 99%, and a residual glass portion present from about 1% to about 20% (by weight of the article).

According to a seventeenth aspect, the article of the sixteenth aspect, wherein the brushite portion includes a plurality of brushite crystals having a crystal size of from about 1 micrometer to about 100 micrometers.

According to the eighteenth aspect, the article of the sixteenth aspect to the seventeenth aspect, wherein the brushite portion and residual glass portion form a component that is at least partially derived from a silica-based glass substrate.

According to the nineteenth aspect, the article of the eighteenth aspect, wherein the silica-based glass substrate is derived from a precursor glass composition including at least about 2% calcium oxide (by weight of oxide).

According to the twentieth aspect, the article of the eighteenth aspect, wherein the silica-based glass substrate is derived from a precursor glass composition including at least about 3% phosphorous pentoxide (by weight of oxide).

According to the twenty-first aspect, the article of the eighteenth aspect, wherein the silica-based glass substrate is derived from a precursor glass composition including at least about 40% silicon dioxide (by weight of oxide).

According to the twenty-second aspect, the article of the eighteenth aspect, wherein the silica-based glass substrate is derived from a precursor glass composition including: SiO₂ from about 40% to about 70%, CaO from about 2% to about 25%, P₂O₅ from about 3% to about 10%, and a total amount of Na₂O plus K₂O of at least about 10% (by weight of oxide).

According to the twenty-third aspect, the article of the eighteenth aspect, wherein the silica-based glass substrate is derived from a precursor glass composition including: SiO₂ from about 43% to about 47%, CaO from about 22.5% to about 26.5%, P₂O₅ from about 4% to about 8%, and Na₂O from about 22.5% to about 26.5% (by weight of oxide).

According to the twenty-fourth aspect, the article of the eighteenth aspect, wherein the silica-based glass substrate is derived from a precursor glass composition including: SiO₂ from about 51% to about 55%, CaO from about 18% to about 22%, P₂O₅ from about 2% to about 6%, K₂O from about 10% to about 14%, and Na₂O from about 4% to about 8% (by weight of oxide).

According to the twenty-fifth aspect, the article of the eighteenth aspect, wherein the silica-based glass substrate is derived from a precursor glass composition including: SiO₂ from about 40% to about 70%, CaO from about 2% to about 30%, P₂O₅ from about 2% to about 15%, MgO from about 0% to about 10%, SrO from about 0% to about 10%, and a total amount of Na₂O plus K₂O of at least about 10% (by weight of oxide).

According to the twenty-sixth aspect, the article of any one of the sixteenth to the twenty-fifth aspects, wherein the article is free, or substantially free, of a hydroxyapatite portion including hydroxyapatite crystals at no more than 1% of the article (by weight). Many variations and modifications may be made to the above-described embodiments of the disclosure without departing substantially from the spirit and various principles of the disclosure. All such modifications and variations are intended to be included herein within the scope of this disclosure and protected by the following claims.

What is claimed is:

1. A method of forming an article, comprising:
immersing a silica-based glass substrate in a liquid medium, wherein the liquid medium comprises a phosphate source at a concentration of at least about 0.1 moles per liter,
converting, during the immersing, at least a portion of the silica-based glass substrate into brushite comprising brushite crystals and a residual glass portion.

2. The method of claim 1, wherein the article comprises from about 1% to about 20% of the residual glass portion (by weight of the article).

3. The method of claim 1, wherein the phosphate source comprises at least one of phosphoric acid, monosodium phosphate, dipotassium phosphate, monopotassium phosphate, and ammonium dihydrogen phosphate.

4. The method of claim 1, wherein the liquid medium comprises a phosphate source at a concentration of from about 0.1 moles per liter to about 5 moles per liter.

5. The method of claim 1, wherein the step of immersing a silica-based glass substrate is conducted for a period of from about 1 day to about 120 days.

6. The method of claim 1, wherein the brushite portion comprises a plurality of brushite crystals having a crystal size of from about 1 micrometer to about 100 micrometers.

7. The method of claim 1, wherein the liquid medium is at a temperature of from about 10° C. to about 85° C. during the step of immersing a silica-based glass substrate.

8. The method of claim 1, wherein the silica-based glass substrate is derived from a precursor glass composition comprising at least one of:
at least about 2% calcium oxide (by weight of oxide);
at least about 3% phosphorous pentoxide (by weight of oxide); and
at least about 40% silicon dioxide (by weight of oxide).

9. The method of claim 1, wherein the silica-based glass substrate is derived from a precursor glass composition comprising:
SiO₂ from about 40% to about 70%,
CaO from about 2% to about 25%,
P₂O₅ from about 3% to about 10%, and
a total amount of Na₂O plus K₂O of at least about 10% (by weight of oxide).

10. The method of claim 1, wherein the silica-based glass substrate is derived from a precursor glass composition comprising:
SiO₂ from about 43% to about 47%,
CaO from about 22.5% to about 26.5%,
P₂O₅ from about 4% to about 8%, and
Na₂O from about 22.5% to about 26.5% (by weight of oxide).

11. The method of claim 1, wherein the silica-based glass substrate is derived from a precursor glass composition comprising:
SiO₂ from about 51% to about 55%,
CaO from about 18% to about 22%,
P₂O₅ from about 2% to about 6%,
K₂O from about 10% to about 14%, and
Na₂O from about 4% to about 8% (by weight of oxide).

12. The method of claim 1, wherein the silica-based glass substrate is derived from a precursor glass composition comprising:
SiO₂ from about 40% to about 70%,
CaO from about 2% to about 30%,
P₂O₅ from about 2% to about 15%,
MgO from about 0% to about 10%,
SrO from about 0% to about 10%, and
a total amount of Na₂O plus K₂O of at least about 10% (by weight of oxide).

13. The method of claim 1, wherein the article is free, or substantially free, of a hydroxyapatite portion comprising hydroxyapatite crystals at no more than 1% of the article (by weight).

14. An article for use as a dental or bone implant, comprising:
a brushite portion that comprises brushite crystals present from about 80% to about 99%, and
a residual glass portion present from about 1% to about 20% (by weight of the article).

15. The article of claim 14, wherein the brushite portion comprises a plurality of brushite crystals having a crystal size of from about 1 micrometer to about 100 micrometers.

16. The article of claim 14, wherein the brushite portion and residual glass portion form a component that is at least partially derived from a silica-based glass substrate.

17. The article of claim 16, wherein the silica-based glass substrate is derived from a precursor glass composition comprising at least one of:
at least about 2% calcium oxide (by weight of oxide);
at least about 3% phosphorous pentoxide (by weight of oxide); and
at least about 40% silicon dioxide (by weight of oxide).

18. The article of claim 16, wherein the silica-based glass substrate is derived from a precursor glass composition comprising:
SiO₂ from about 40% to about 70%,
CaO from about 2% to about 25%,
P₂O₅ from about 3% to about 10%, and
a total amount of Na₂O plus K₂O of at least about 10% (by weight of oxide).

19. The article of claim 16, wherein the silica-based glass substrate is derived from a precursor glass composition comprising:
SiO₂ from about 40% to about 70%,
CaO from about 2% to about 30%,
P₂O₅ from about 2% to about 15%,
MgO from about 0% to about 10%,
SrO from about 0% to about 10%, and
a total amount of Na₂O plus K₂O of at least about 10% (by weight of oxide).

20. The article of claim 14, wherein the article is free, or substantially free, of a hydroxyapatite portion comprising hydroxyapatite crystals at no more than 1% of the article (by weight).
